(19) United States
(12) Patent Application Publication
(10) Pub. No.: US 2018/0332271 A1
(43) Pub. Date: Nov. 15, 2018

(54) IMAGE PICKUP APPARATUS, THREE-DIMENSIONAL ENDOSCOPE AND THREE-DIMENSIONAL ENDOSCOPE SYSTEM

(71) Applicant: OLYMPUS CORPORATION, Tokyo (JP)

(72) Inventor: Susumu AONO, Tokyo (JP)

(73) Assignee: OLYMPUS CORPORATION, Tokyo (JP)

(21) Appl. No.: 16/038,233

(22) Filed: Jul. 18, 2018

Related U.S. Application Data
(63) Continuation of application No. PCT/JP2016/078634, filed on Sep. 28, 2016.

(30) Foreign Application Priority Data
Feb. 12, 2016 (JP) 2016-024842

(51) Int. Cl. H04N 13/207; A61B 1/00; A61B 1/045

(52) U.S. Cl. CPC H04N 13/207; A61B 1/045; A61B 1/00193

(57) ABSTRACT

An image pickup apparatus is provided with: an objective optical system; at least one image pickup device configured to pick up two subject images; at least one moving lens portion configured to change optical characteristics of the two subject images; and at least one actuator configured to drive the moving lens portion.

### CROSS REFERENCE TO RELATED APPLICATION

This application is a continuation application of PCT/JP2016/078634 filed on Sep. 28, 2016 and claims benefit of Japanese Application No. 2016-024842 filed in Japan on Feb. 12, 2016, the entire contents of which are incorporated herein by this reference.

### BACKGROUND OF THE INVENTION

### 1. Field of the Invention

The present invention relates to a three-dimensional endoscope and a three-dimensional endoscope system provided with an image pickup apparatus configured to pick up a three-dimensional image.

### 2. Description of the Related Art

In order to observe a place which is difficult to observe, such as an interior of a living body or an inside of a structure, an endoscope that can be introduced into an inside of a living body or a structure from outside and that is provided with an optical unit such as an image pickup unit for picking up an optical image is used, for example, in a medical field or an industrial field.

Among such endoscopes, a twin-lens 3D endoscope that combines two observation images to generate a 3D image so that a subject or object can be three-dimensionally viewed has appeared.

For example, Japanese Patent Application Laid-Open Publication No. 8-94965 proposes an image pickup apparatus for endoscope in which a convergence angle of an image pickup portion configured to pick up a three-dimensional image can be easily changed on an image pickup side.

### SUMMARY OF THE INVENTION

A three-dimensional endoscope of one aspect of the present invention is provided with: a first objective optical system including a first moving lens portion; a second objective optical system including a second moving lens portion; one or two image pickup devices configured to pick up a first subject image formed by the first objective optical system and a second subject image formed by the second objective optical system; an actuator configured to contract or extend in a longitudinal direction; and one coupling member connected to an end portion of the actuator and coupled with the first moving lens portion and the second moving lens portion, the coupling member being configured to, in response to contraction or extension of the actuator, cause the first moving lens portion and the second moving lens portion to simultaneously move.

A three-dimensional endoscope system of one aspect of the present invention is provided with: an image pickup apparatus including: an objective optical system; at least one image pickup device configured to pick up two subject images; two moving lens portions configured to change optical characteristics of the two subject images; and at least two actuators configured to drive the moving lens portions; a three-dimensional endoscope including an insertion portion including a distal end portion incorporating the image pickup apparatus; and a control portion configured to calculate a difference between image positions of the two subject images acquired by the image pickup device and drive-control the two actuators to correct positions of the two moving lens portions.

### BRIEF DESCRIPTION OF THE DRAWINGS

FIG. 1 is a perspective view showing a configuration of a three-dimensional endoscope system according to a first embodiment;

FIG. 2 is a cross-sectional view showing a configuration of an image pickup apparatus incorporated in a distal end portion according to the first embodiment;

FIG. 3 is a cross-sectional view showing a configuration of an image pickup apparatus incorporated in a distal end portion according to a first modification of the first embodiment;

FIG. 4 is a cross-sectional view showing a configuration of an image pickup apparatus incorporated in a distal end portion according to a second modification of the first embodiment;

FIG. 5 is a cross-sectional view showing a configuration of an image pickup apparatus incorporated in a distal end portion according to a third modification of the first embodiment;

FIG. 6 is a cross-sectional view showing a configuration of an image pickup apparatus incorporated in a distal end portion according to a fourth modification of the first embodiment;

FIG. 7 is a block diagram mainly showing a control configuration of a three-dimensional endoscope and a video processor in a three-dimensional endoscope system according to a second embodiment; and

FIG. 8 is a block diagram mainly showing a control configuration of a three-dimensional endoscope and a video processor in a three-dimensional endoscope system according to a modification of the second embodiment.

### DETAILED DESCRIPTION OF THE PREFERRED EMBODIMENTS

The present invention will be described below. Note that drawings based on each embodiment are schematic in the description below, and relationships between thickness and width of each portion, thickness ratios among respective portions and the like are different from actual ones; and, among the drawings, portions having different mutual dimensional relationships or ratios may be included.

### First Embodiment

First, a first embodiment of the present invention will be described.

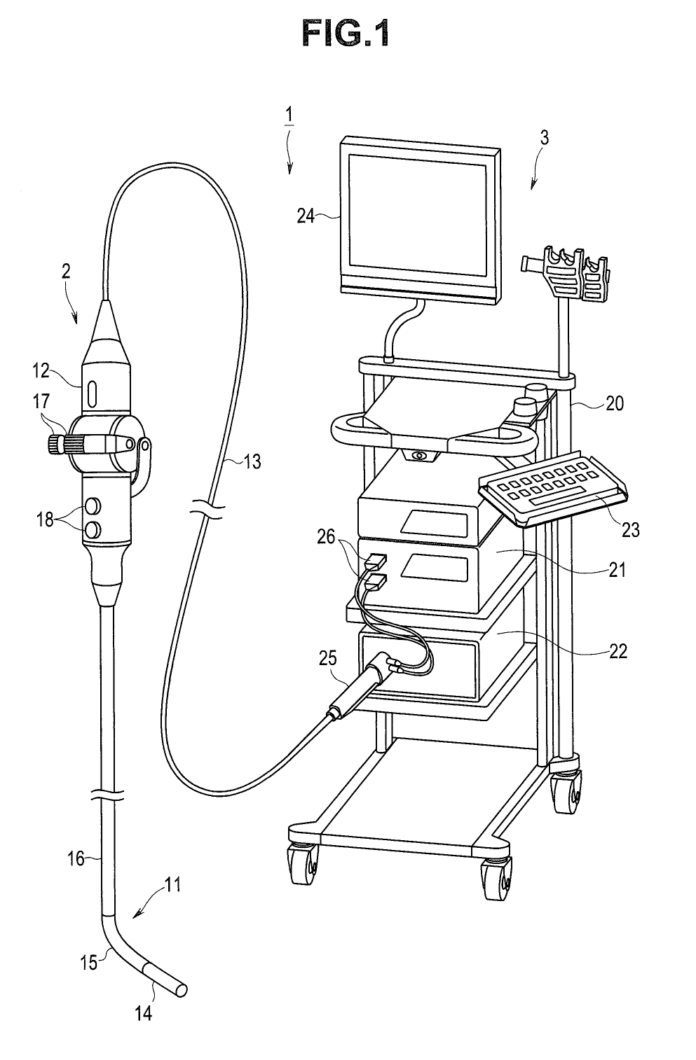

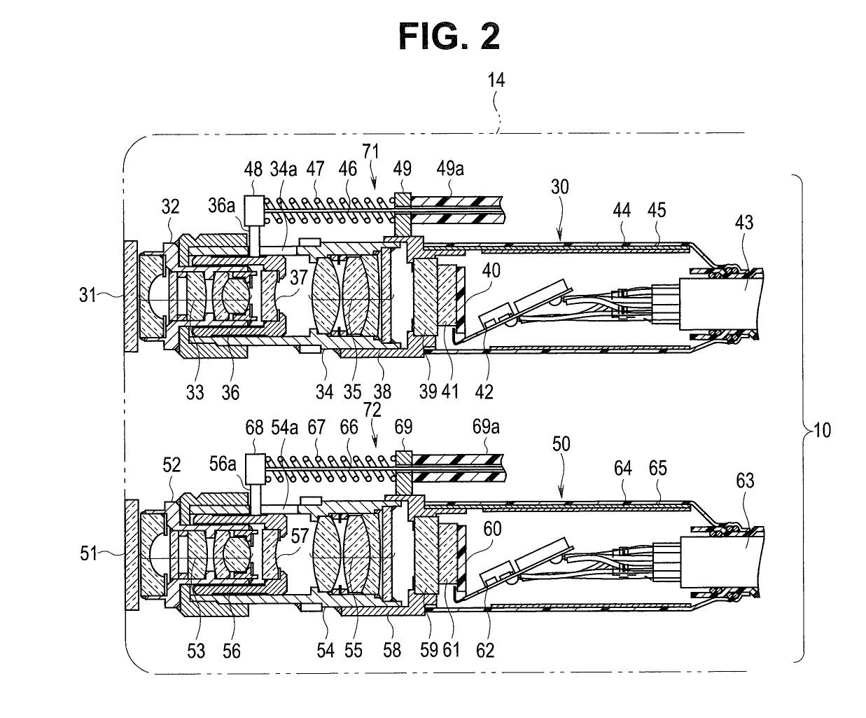

Drawings are related to the first embodiment of the present invention. FIG. 1 is a perspective view showing an overall configuration of a three-dimensional endoscope system; and FIG. 2 is a cross-sectional view showing a configuration of an image pickup apparatus incorporated in a distal end portion.

As shown in FIG. 1, a three-dimensional endoscope system 1 is configured including a three-dimensional endoscope 2 and a video system center 3. Note that the three-dimensional endoscope 2 here is a twin-lens three-dimensional endoscope (also referred to as a 3D endoscope) configured to combine two observation images to generate a 3D image so that a subject can be three-dimensionally viewed.

The three-dimensional endoscope 2 is configured including an insertion portion 11, an operation portion 12 arranged being connected to a proximal end of the insertion portion 11 and a universal cord 13 extending from the operation portion 12.

The insertion portion 11 is configured with a distal end portion 14, a bending portion 15 and a rigid tube portion 16 arranged being connected in order from a distal end. Note that though the three-dimensional endoscope 2 here is exemplified as a so-called rigid three-dimensional endoscope that is used for surgery, in which the insertion portion 11 includes the rigid tube portion 16, the three-dimensional endoscope 2 is not limited to the rigid three-dimensional endoscope but may be a so-called flexible three-dimensional endoscope in which the insertion portion 11 has flexibility.

The distal end portion 14 is provided with two observation windows and an illumination window on a distal end face. Two observation lights incident from the two observation windows are detected by an image sensor such as a CCD or a CMOS provided on an image pickup apparatus via a plurality of objective optical systems (none of the components is shown).

The operation portion 12 includes two bending operation levers 17 configured to operate the bending portion 15 of the insertion portion 11 in an up-and-down direction (a UD direction) of an observation image as a first direction and a right-and-left direction (an RL direction) of the observation image as a second direction different from the first direction, here, a second direction substantially orthogonal to the first direction, in response to an operation of an operator who is a doctor.

The operation portion 12 is provided with buttons 18 for operating an observation image, such as tele/wide switching and release switches.

The video system center 3 is configured mainly including a video processor 21 as a control device configured to control functions of various three-dimensional endoscopes 2 mounted on a trolley 20, a light source device 22 incorporating a light source of illuminating light to be radiated toward a subject from the illumination window of the distal end portion 14 of the three-dimensional endoscope 2, a keyboard 23 and a monitor 24.

The video processor 21, which is a control device, performs lighting control of the light source device 22 and performs image processing of an image of a subject photographed through the three-dimensional endoscope 2 to display the image on the monitor 24.

Note that an extension end of the universal cord 13 of the three-dimensional endoscope 2 is provided with a light source connector 25 to be detachably connected to the light source device 22.

Here, two electrical cables 26 are arranged extending from the light source connector 25, and each of extension ends of the electrical cables 26 is provided with an electrical connector 27 to be detachably connected to the video processor 21.

Note that, as for internal components of the three-dimensional endoscope 2, which is a three-dimensional endoscope, detailed description of the components will be omitted because the components are similar to conventional components.

Next, a configuration of an image pickup apparatus 10 of the present embodiment will be described below in detail.

As shown in FIG. 2, the image pickup apparatus 10 is provided with a first image pickup unit 30 and a second image pickup unit 50, which are two image pickup portions having the same configuration.

The first image pickup unit 30 is arranged behind a first observation window 31 arranged on the distal end face of the distal end portion 14. The second image pickup unit 50 is arranged behind a second observation window 51 arranged on the distal end face of the distal end portion 14.

That is, the three-dimensional endoscope 2 of the present embodiment is a three-dimensional endoscope provided with two image pickup units, the first image pickup unit 30 and the second image pickup unit 50. Note that, here, the first image pickup unit 30 constitutes an observation-image-for-right-eye forming portion and the second image pickup unit 50 constitutes an observation-image-for-left-eye forming portion.

The first image pickup unit 30 and second image pickup unit 50 are provided with front group lens frames 32 and 52 which are first fixed lens frames, front group lenses 33 and 53 which are objective optical systems held by the front group lens frames 32 and 52, rear group lens frames 34 and 54 which are second fixed lens frames to be fitted to the front group lens frames 32 and 52, and front group lenses 35 and 55 which are objective optical systems held by the rear group lens frames 34 and 54, respectively.

The first image pickup unit 30 and the second image pickup unit 50 are provided with moving lens frames 36 and 56 which are moving lens portions which advancingly or retreatingly move forward or backward in the rear group lens frames 34 and 54, respectively, and moving lenses 37 and 57 which are objective optical systems are held by the moving lens frames 36 and 56.

Note that the respective first image pickup unit 30 and second image pickup unit 50 are configured to be capable of changing an optical characteristic, here, changing a view angle between tele and wide by the moving lens frames 36 and 56 holding the moving lenses 37 and 57 moving forward or backward.

Image pickup device holding frames 38 and 58 are fitted behind the rear group lens frames 34 and 54, and image pickup devices 40 and 60, such as a CCD or a CMOS, are fixed to transparent glasses 39 and 59 held by the image pickup device holding frames 38 and 58 via cover glasses 41 and 61.

Image pickup device boards 42 and 62 mounted with electronic parts and the like are electrically connected to the image pickup devices 40 and 60. A plurality of wires are connected to image pickup device boards 42 and 62, and image pickup cables 43 and 63, which are the plurality of wires collected together, are arranged extending rearward.

Note that the image pickup cables 43 and 63 are arranged in the insertion portion 11, the operation portion 12, the universal cord 13 and the light source connector 25 and connected to the electrical connectors 27 provided on the two electrical cables 26.

Reinforcing frames 44 and 64 are fitted behind the image pickup device holding frames 38 and 58, and thermal contraction tubes 45 and 65 are provided in a manner of covering distal end parts of the image pickup cables 43 and 63 together with the reinforcing frames 44 and 64.

Note that filling material for protecting the image pickup devices 40 and 60, the image pickup device boards 42 and 62 and the like, such as adhesive, is arranged in the reinforcing frames 44 and 64.

The first image pickup unit 30 and the second image pickup unit 50 of the present embodiment are provided with actuators 71 and 72, which are view angle changing portions configured to drive the moving lens frames 36 and 56 to advance or retreat forward or backward, respectively.

The actuators 71 and 72 here drive the moving lens frames 36 and 56 using shape memory alloy (SMA) wires 46 and 66 and spring members 47 and 67.

Distal ends of the shape memory alloy (SMA) wires 46 and 66 are connected to block bodies 48 and 68 provided at distal ends of rod-shaped connection bodies 36a and 56a extending in an outer diameter direction from the moving lens frames 36 and 56.

Note that, in the rear group lens frames 34 and 54, slits 34a and 54a are formed to guide the connection bodies 36a and 56a to advance straight and extend the connection bodies 36a and 56a in the outer diameter direction.

The shape memory alloy (SMA) wires 46 and 66 are arranged extending rearward and inserted into spring holders 49 and 69 provided on the image pickup device holding frames 38 and 58. Insulation tubes 49a and 69a are connected to the spring holders 49 and 69, and the shape memory alloy (SMA) wires 46 and 66 are arranged in the insulation tubes 49a and 69a.

The spring members 47 and 67 are fitted on the shape memory alloy (SMA) wires 46 and 66 and arranged between the block bodies 48 and 68 and the spring holders 49 and 69. The spring members 47 and 67 energize the block bodies 48 and 68 forward.

Note that although not shown in the drawings, the shape memory alloy (SMA) wires 46 and 66 are fixed at proximal ends of the insulation tubes 49a and 69a.

The shape memory alloy (SMA) wires 46 and 66 are set, for example, so as to be contracted when being heated and extended when being cooled, and held in the insulation tubes 49a and 69a in an extendable and contractible state.

Heat sources such as Peltier elements not shown are arranged together with the shape memory alloy (SMA) wires 46 and 66. The heat sources can heat or cool the shape memory alloy (SMA) wires 46 and 66 in response to an operation of a common view angle change switch among the buttons 18 provided on the operation portion 12.

That is, the two actuators 71 and 72 of the image pickup apparatus 10 are configured to synchronously drive the two moving lens frames 36 and 56 provided in the first image pickup unit 30 and the second image pickup unit 50, respectively, in response to an operation of the common view angle change switch among the buttons 18 so that the two moving lens frames 36 and 56 advancingly or retreatingly move forward or backward synchronously.

Note that the shape memory alloy (SMA) wires 46 and 66 are not limited to shape memory alloy (SMA) wires adopting a method of causing extension or contraction by heating or cooling using heat sources such as Peltier elements. For example, it is also possible to adopt a method such as heating shape memory alloy by energization to cause the shape memory alloy to be contracted.

That is, in the first image pickup unit 30 and the second image pickup unit 50, the block bodies 48 and 68 are pulled rearward against energizing forces of the spring members 47 and 67 by the shape memory alloy (SMA) wires 46 and 66 of the actuators 71 and 72 of the first image pickup unit 30 and the second image pickup unit 50, respectively, being heated and contracted.

In the first image pickup unit 30 and the second image pickup unit 50, the block bodies 48 and 68 receiving the energizing forces of the spring members 47 and 67 are pushed forward by the shape memory alloy (SMA) wires 46 and 66 of the actuators 71 and 72 of the first image pickup unit 30 and the second image pickup unit 50, respectively, being cooled and extended.

Thereby, in the first image pickup unit 30 and the second image pickup unit 50, the view angle can be changed to tele or wide by the moving lens frames 36 and 56 connected to the block bodies 48 and 68 via the connection bodies 36a and 56a, respectively, moving forward or backward.

Note that the forward and backward positions of the moving lens frames 36 and 56 for changing the view angle between tele and wide are determined by lens design of the first image pickup unit 30 and the second image pickup unit 50 of the image pickup apparatus 10 and are not especially limited.

Thus, the three-dimensional endoscope 2, which is a three-dimensional endoscope of the present embodiment, is such that a user can adjust or change a view angle of a 3D image to be acquired by the image pickup apparatus 10 provided with two image pickup portions, the first image pickup unit 30 and the second image pickup unit 50, as he desires.

For example, if it is desired to decrease (weaken) a three-dimensional appearance of a subject, the subject can be observed on a tele side where the view angle is decreased, and the object displayed on the monitor 24 is displayed large.

At this time, the user can adjust the subject displayed on the monitor 24 to a desired size by moving the distal end portion 14 of the three-dimensional endoscope 2 away from the subject. By the three-dimensional appearance of the 3D image being decreased, a feeling of oppression of a treatment instrument and the like treating the subject can be reduced, and workability is improved.

On the other hand, for example, if it is desired to increase (strengthen) the three-dimensional appearance of the subject, the subject can be observed on a wide side where the view angle is increased, and the object displayed on the monitor 24 is displayed small.

At this time, the user can adjust the subject displayed on the monitor 24 to a desired size by moving the distal end portion 14 of the three-dimensional endoscope 2 close to the subject. Since it becomes easier to perform examination and treatment because of the subject being displayed on the monitor 24 more three-dimensionally especially when a distance between the distal end portion 14 of the three-dimensional endoscope 2 and the subject is relatively large, workability is improved by the three-dimensional appearance of the 3D image being increased (strengthened).

As described above, by being provided with the image pickup apparatus 10 including two image pickup portions, the first image pickup unit 30 and the second image pickup unit 50, which are capable of changing a view angle of a subject image to be acquired, the three-dimensional endoscope 2 of the present embodiment is in a configuration in which the user can adjust and change a 3D image to a desired three-dimensional appearance, and it is possible to cause workability of examination, treatment and the like for a subject to be improved.

Note that the image pickup apparatus 10 may be in a configuration in which not only a view angle but also an optical characteristic for focus adjustment can be changed, by the two moving lens frames 36 and 56 advancingly or retreatingly moving forward or backward synchronously by the two actuators 71 and 72 of the first image pickup unit 30 and the second image pickup unit 50.

### First Modification

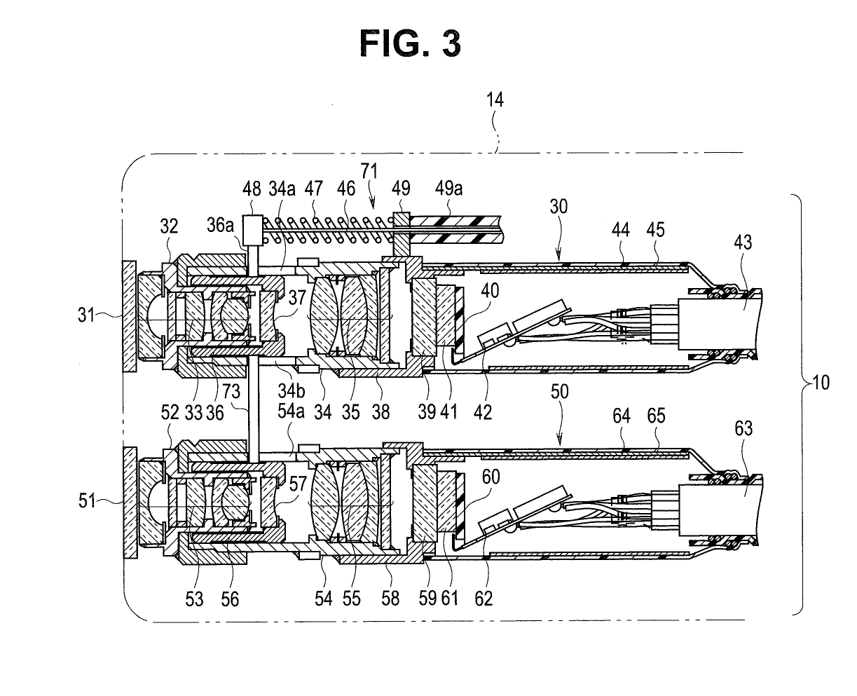

FIG. 3 is a cross-sectional view showing a configuration of an image pickup apparatus incorporated in a distal end portion according to a first modification.

As shown in FIG. 3, the image pickup apparatus 10 may be in a configuration of advancingly or retreatingly moving the moving lens frames 36 and 56 of the first image pickup unit 30 and the second image pickup unit 50 forward or backward by one actuator 71.

More specifically, the image pickup apparatus 10 of the present modification is provided with a rod-shaped coupling member 73 coupling the moving lens frame 36 of the first image pickup unit 30 and the moving lens frame 56 of the second image pickup unit 50, and a slit 34b for guiding the coupling member 73 to advance straight and causing the coupling member 73 to extend in the outer diameter direction is formed in the rear group lens frame 34 of the first image pickup unit 30.

Note that the coupling member 73 is connected to the moving lens frame 56 of the second image pickup unit 50, extends in the outer diameter direction via the slit 54a of the rear group lens frame 54 of the second image pickup unit 50, and is connected to the rear group lens frame 34 of the first image pickup unit 30.

The image pickup apparatus 10 of the present modification configured as described above can cause the moving lens frame 36 of the first image pickup unit 30 and the moving lens frame 56 of the second image pickup unit 50 to advancingly or retreatingly move forward or backward by the one actuator 71.

Thereby, since an amount of forward or backward displacement of the moving lens frame 36 of the first image pickup unit 30 and the moving lens frame 56 of the second image pickup unit 50 is always constant, the image pickup apparatus 10 is in a configuration in which a view angle of a subject image to be acquired can be stably changed.

### Second Modification

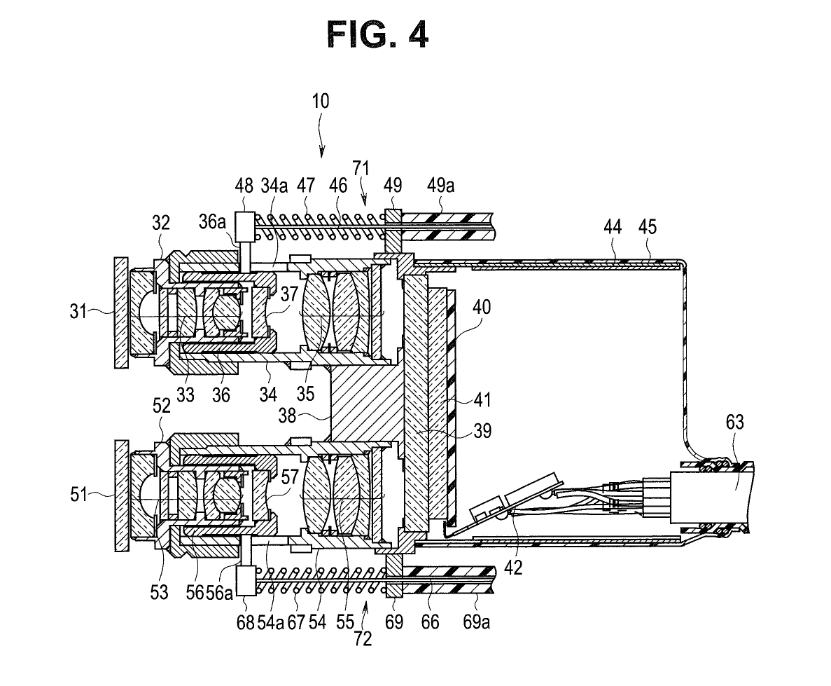

FIG. 4 is a cross-sectional view showing a configuration of an image pickup apparatus incorporated in a distal end portion according to a second modification.

As shown in FIG. 4, the image pickup apparatus 10 may be in a configuration in which the two moving lenses 37 and 57, which are moving optical systems, are provided, and one image pickup device holding frame 38 is fitted to the rear group lens frames 34 and 54, and two images are acquired by one image pickup device 40.

Note that though the configuration of advancingly or retreatingly moving the two moving lens frames 36 and 56 forward or backward by the two actuators 71 and 72 is shown in FIG. 4, a configuration is not limited to the configuration. The configuration of causing the two moving lens frames 36 and 56 to advancingly or retreatingly move forward or backward by the one actuator 71 as exemplified in the first modification may be adopted.

### Third Modification

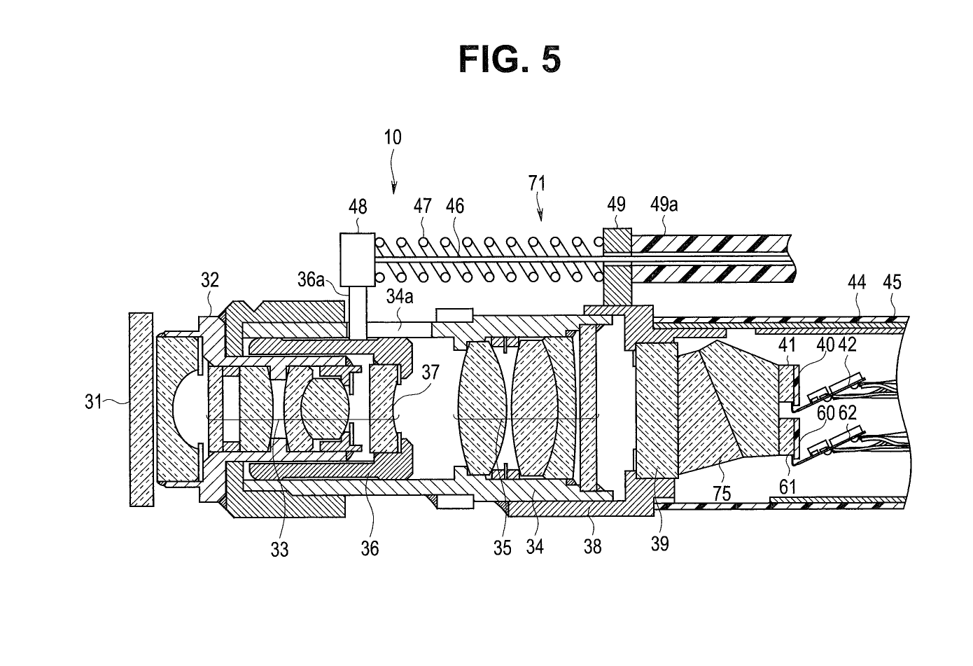

FIG. 5 is a cross-sectional view showing a configuration of an image pickup apparatus incorporated in a distal end portion according to a third modification.

As shown in FIG. 5, the image pickup apparatus 10 may be in a configuration in which only the moving lens 37, which is one moving optical system, is provided, and two images are acquired by the two image pickup devices 40 and 60.

Note that the image pickup apparatus 10 of the present modification is provided with a prism unit 75 behind the transparent glass 39 of the image pickup device holding frame 38 and is configured such that two subject images enter the two image pickup devices 40 and 60 by the prism unit 75.

Thus, since the image pickup apparatus 10 of the present modification changes a view angle of a subject image to be acquired by one moving lens frame 36, view angle displacement between a subject image for right eye and a subject image for left eye to be acquired can be prevented.

### Fourth Modification

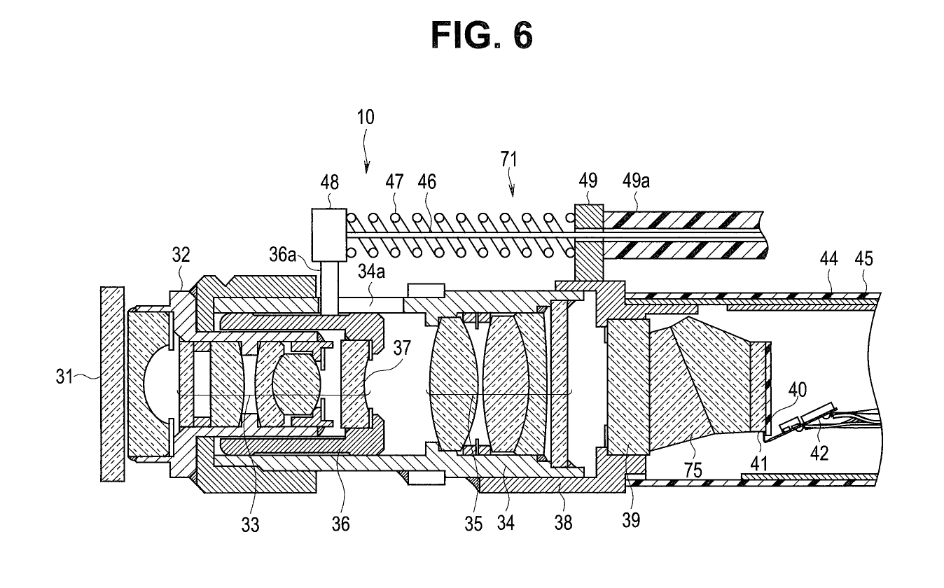

FIG. 6 is a cross-sectional view showing a configuration of an image pickup apparatus incorporated in a distal end portion according to a fourth modification.

As shown in FIG. 6, the image pickup apparatus 10 may be in a configuration in which only the moving lens 37, which is one moving optical system, is provided, and two images are acquired by one image pickup device 40.

Note that the image pickup apparatus 10 of the present modification is also provided with the prism unit 75 behind the transparent glass 39 of the image pickup device holding frame 38 and is configured such that two subject images enter the one image pickup device 40 by the prism unit 75.

Similarly to the third modification, since the image pickup apparatus 10 of the present modification also changes a view angle of a subject image to be acquired by one moving lens frame 36, view angle displacement between a subject image for right eye and a subject image for left eye to be acquired can be prevented.

### Second Embodiment

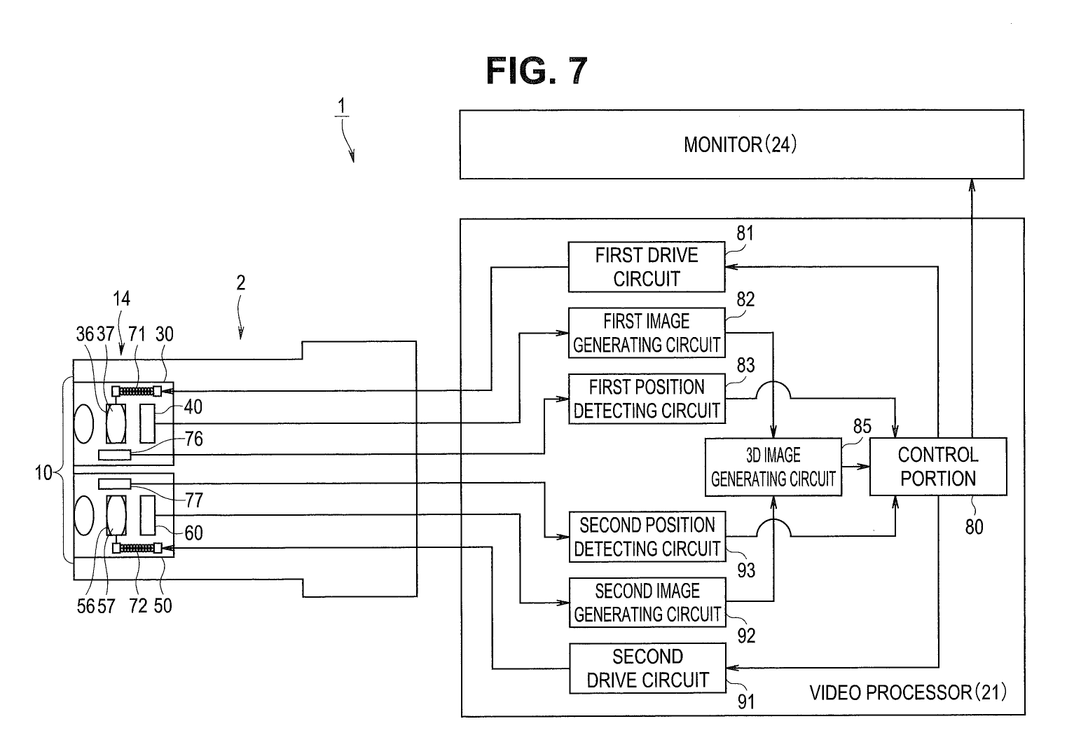

Next, a second embodiment of the present invention will be described. FIG. 7 is a block diagram mainly showing a control configuration of a three-dimensional endoscope and a video processor in a three-dimensional endoscope system.

Note that, in the present embodiment, the same reference numerals are used for the respective components described in the first embodiment described above, and detailed description of the components will be omitted.

As shown in FIG. 7, in the image pickup apparatus 10 incorporated in the distal end portion 14 of the three-dimensional endoscope 2 of the present embodiment, position detection sensors 76 and 77 as position detecting portions configured to detect positions of the moving lens frames 36 and 56 holding the moving lenses 37 and 57 are arranged in the first image pickup unit 30 and the second image pickup unit 50, respectively.

Note that the position detection sensors 76 and 77 are provided near the moving lens frames 36 and 56, and are moving lens frame position detecting portions such as potentiometers, encoders and longitudinal position detection sensors.

In the three-dimensional endoscope system 1, the video processor 21 to which the three-dimensional endoscope 2 is connected is provided with a control portion 80 configured to drive-control the first image pickup unit 30 and the second image pickup unit 50 of the image pickup apparatus 10.

The video processor 21 is provided with a first drive circuit 81 configured to drive the actuator 71 of the first image pickup unit 30, a first image generating circuit 82 to which an image pickup signal from the image pickup device 40 of the first image pickup unit 30 is inputted, and a first position detecting circuit 83 to which a detection signal from the position detection sensor 76 of the first image pickup unit 30 is inputted.

The video processor 21 is provided with a second drive circuit 91 configured to drive the actuator 72 of the second image pickup unit 50, a second image generating circuit 92 to which an image pickup signal from the image pickup device 60 of the second image pickup unit 50 is inputted, and a second position detecting circuit 93 to which a detection signal from the position detection sensor 77 of the second image pickup unit 50 is inputted.

The video processor 21 incorporates a 3D image generating circuit 85 configured to, when two images are inputted from the first image generating circuit 82 and the second image generating circuit 92, combine the two images to generate a 3D image. From the 3D image generating circuit 85, a video signal of the 3D image is outputted to the monitor 24 via the control portion 80. In this way, the 3D image of a subject photographed by the image pickup apparatus 10 is displayed on the monitor 24.

Position information about the moving lens frame 36 of the first image pickup unit 30 and the moving lens frame 56 of the second image pickup unit 50 is inputted to the control portion 80 from the first position detecting circuit 83 and the second position detecting circuit 93.

The control portion 80 controls the first drive circuit 81 and the second drive circuit 91 based on the position information about the respective moving lens frames 36 and 56 to drive the respective actuators 71 and 72. Note that although not shown in the drawings, a signal is inputted to the control portion 80 from the view angle change switch provided on the operation portion 12, and the control portion 80 outputs control signals based on the signal to the first drive circuit 81 and the second drive circuit 91.

Thus, at the time of drive-controlling the respective actuators 71 and 72 to move the moving lens frame 36 of the first image pickup unit 30 and the moving lens frame 56 of the second image pickup unit 50 forward or backward, the control portion 80 of the video processor 21 calculates, based on the position information about the respective moving lens frames 36 and 56 detected by the position detection sensors 76 and 77 and inputted from the first position detecting circuit 83 and the second position detecting circuit 93, a difference between the respective positions, corrects the difference and outputs the control signals to the first drive circuit 81 and the second drive circuit 91 so that the forward and backward movement positions (forward and backward positions in the direction along a photographing optical axis) of the respective moving lens frames 36 and 56 correspond to each other.

Then, the first drive circuit 81 and the second drive circuit 91 drive the respective actuators 71 and 72 to drive the respective moving lens frames 36 and 56, based on the control signal from the control portion 80.

Thus, the three-dimensional endoscope system 1 of the present embodiment calculates, from position information about the moving lens frame 36 of the first image pickup unit 30 and the moving lens frame 56 of the second image pickup unit 50 for right eye and for left eye, a difference between respective positions and performs control to perform correction so that movement positions of the respective moving lens frames 36 and 56 correspond to each other, by the video processor 21.

Thereby, in addition to the operation and effect described in the first embodiment, the three-dimensional endoscope system 1 can generate a 3D image in which view angles of subject images to be acquired by the first image pickup unit 30 and the second image pickup unit 50 of the image pickup apparatus 10 always mutually correspond.

Note that though the image pickup apparatus 10 including the two image pickup devices 40 and 60 is exemplified in the present embodiment, the present embodiment can be also applied to such a three-dimensional endoscope system 1 in which two subject images are acquired by one image pickup device to generate a 3D image.

### Modification

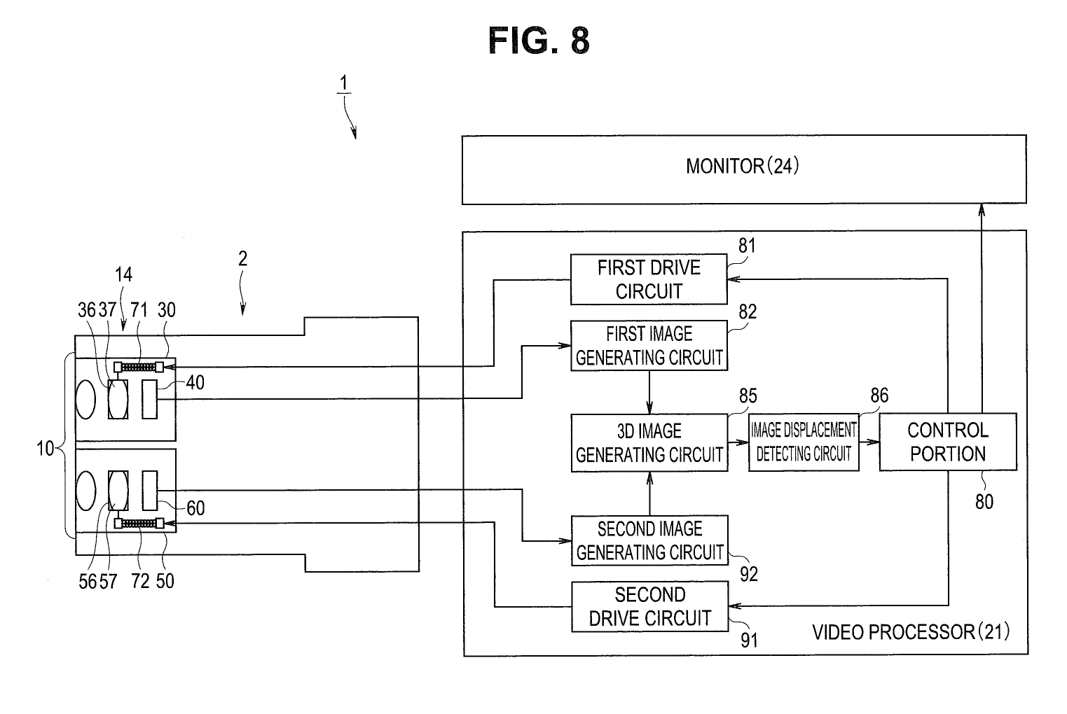

FIG. 8 is a block diagram mainly showing a control configuration of a three-dimensional endoscope and a video processor in a three-dimensional endoscope system of a modification.

Note that, as shown in FIG. 8, the three-dimensional endoscope system 1 may detect a plurality of features of two subject images acquired by the first image pickup unit 30 and the second image pickup unit 50 and perform control to correct movement positions of the respective moving lens frames 36 and 56, by the video processor 21, without being provided with the position detection sensors 76 and 77 on the image pickup apparatus 10 side of the three-dimensional endoscope 2.

More specifically, the video processor 21 is provided with an image displacement detecting circuit 86 as an image displacement detecting portion to which an image signal from the 3D image generating circuit 85 is inputted, and a correction signal from the image displacement detecting circuit 86 is inputted to the control portion 80.

The image displacement detecting circuit 86 calculates, for example, a difference between sizes or positions of two subject images acquired by the first image pickup unit 30 and the second image pickup unit 50, from a 3D image from the 3D image generating circuit 85, and outputs to the control portion 80a position correction instruction signal for causing forward and backward movement positions (forward and backward positions in the direction along the photographing optical axis) of the respective moving lens frames 36 and 56 to correspond to each other, in which the difference is corrected.

Then, based on the position correction instruction signal from the image displacement detecting circuit 86, the control portion 80 outputs control signals to the first drive circuit 81 and the second drive circuit 91 so that the forward and backward movement positions (the forward and backward positions in the direction along the photographing optical axis) of the respective moving lens frames 36 and 56 correspond to each other.

Then, the first drive circuit 81 and the second drive circuit 91 drive the respective actuators 71 and 72 to drive the respective moving lens frames 36 and 56, based on the control signal from the control portion 80.

Thus, the three-dimensional endoscope system 1 of the present modification calculates, from subject images acquired by the first image pickup unit 30 and the second image pickup unit 50 for right eye and for left eye, a difference between positions of the respective moving lens frames 36 and 56, and performs control to perform correction so that movement positions of the respective moving lens frames 36 and 56 correspond to each other, by the video processor 21.

Even when such a configuration is adopted, the three-dimensional endoscope system 1 can generate such a 3D image that view angles of subject images to be acquired by the first image pickup unit 30 and the second image pickup unit 50 of the image pickup apparatus 10 always mutually correspond.

Note that, in the present modification, though the image pickup apparatus 10 including the two image pickup devices 40 and 60 is exemplified, the present modification can be also applied to such a three-dimensional endoscope system 1 in which two subject images are acquired by one image pickup device to generate a 3D image.

The present invention described in each of the embodiments described above is not limited to the embodiments and modifications. Additionally, various modifications can be practiced within a range not departing from the spirit of the invention at a practice stage. The above embodiments include inventions at various stages, and various inventions can be extracted by an appropriate combination among a plurality of constituent features disclosed.

For example, even if some constituent features are deleted from all the constituent features shown in an embodiment, the configuration from which the constituent features have been deleted can be extracted as an invention if the stated problem can be solved and the stated effect can be obtained.

According to the present invention, it is possible to provide an image pickup apparatus for endoscope, a three-dimensional endoscope and a three-dimensional endoscope system in which a user can adjust and change a 3D image to a desired three-dimensional appearance, and workability of examination, treatment and the like for a subject is improved.

What is claimed is:

1. A three-dimensional endoscope system comprising:
an image pickup apparatus comprising:
an objective optical system;
at least one image pickup device configured to pick up two subject images;
two moving lens portions configured to change optical characteristics of the two subject images; and
at least two actuators configured to drive the moving lens portions;
a three-dimensional endoscope comprising an insertion portion comprising a distal end portion incorporating the image pickup apparatus; and
a control portion configured to calculate a difference between image positions of the two subject images acquired by the image pickup device and drive-control the two actuators to correct positions of the two moving lens portions.

2. A three-dimensional endoscope comprising:
a first objective optical system comprising a first moving lens portion;
a second objective optical system comprising a second moving lens portion;
one or two image pickup devices configured to pick up a first subject image formed by the first objective optical system and a second subject image formed by the second objective optical system;
an actuator configured to contract or extend in a longitudinal direction; and
one coupling member connected to an end portion of the actuator and coupled with the first moving lens portion and the second moving lens portion, the coupling member being configured to, in response to contraction or extension of the actuator, cause the first moving lens portion and the second moving lens portion to simultaneously move.

3. The three-dimensional endoscope according to claim 2, wherein the actuator is a shape memory alloy wire.
